(19) United States
(12) Patent Application Publication
(10) Pub. No.: US 2024/0307123 A1
(43) Pub. Date: Sep. 19, 2024

(54) ROBOTIC CATHETER AND AUTOMATIC NAVIGATION SYSTEM

(71) Applicant: ROBOCATH, ROUEN (FR)

(72) Inventor: Pierre SCHEGG, ROUEN (FR)

(21) Appl. No.: 18/576,649

(22) PCT Filed: Jun. 28, 2022
(86) PCT No.: PCT/EP2022/067777

§ 371 Date: Jan. 4, 2024

(30) Foreign Application Priority Data
Jul. 7, 2021 (FR) 2107331

(51) Int. Cl. A61B 34/10; A61B 34/30; A61B 34/32

(52) U.S. Cl. CPC A61B 34/10; A61B 34/32; A61B 2034/102; A61B 2034/105; A61B 2034/301; A61B 2034/303

(57) ABSTRACT

This relates to a catheter robot including an automatic navigation system, for an elongate flexible medical instrument with a free distal end, implementing an automatic navigation method including successively: a step of creating a modeling: of the elongate flexible medical instrument, of a blood circulatory system, of the interaction thereof, a step of determining: a path to be followed, between the start point and the point of arrival, a step of planning a sequence of commands for moving the elongate flexible medical instrument, obtained by a tree search procedure, a step of implementing the planned sequence of commands, with compensation for any differences with respect to the determined path along the modeled system, by closed-loop regulation.

### FIELD OF THE INVENTION

The invention relates to a catheter robot, an automatic navigation system, and a method for the automatic navigation of an elongate flexible medical instrument that is a catheter or a catheter guide, with free distal end.

### Technological Background of the Invention

According to a first prior art, for example described in the patent application US2020/0129740, an automatic navigation system in a robot for an elongate flexible medical instrument is known. However, in this document, the automatic navigation for this elongate flexible medical instrument is solely envisaged, and it is not specified which algorithm can control the movements of this elongate flexible medical instrument.

According to a second prior art, for example described in the English-language article having the title “Automatic control of cardiac ablation catheter with deep reinforcement learning method”, an automatic navigation system in a robot for an elongate flexible medical instrument is known, but this elongate flexible medical instrument is of a particular type that has a distal end that is steerable (“steerable catheter”), which is in fact an ablation catheter with steerable tip, which naturally makes the automatic navigation easier to achieve for this elongate flexible medical instrument having a steerable distal end. In addition, to determine the path to be followed by this elongate flexible medical instrument having a steerable distal end, the navigation method uses a deep network reinforcement learning method (DQN, standing for “Deep Q-Learning”), said method having two drawbacks which are first of all the need for prior training and next the difficulty of generalizing to any type of artery topology and geometry of a patient.

According to a third prior art, for example described in the English-language article having the title “Reinforcement learning for guidewire navigation in coronary phantom”, an automatic navigation system in a robot for an elongate flexible medical instrument is known, but this navigation is implemented only in a two-dimensional (2D) space, the possibility of adapting it for automatic navigation in a three-dimensional (3D) space remaining purely hypothetical, which does not make this automatic navigation a good candidate for moving in three dimensions (3D) in a real blood circulatory system. In addition, for determining the path to be followed by this elongate flexible medical instrument having a steerable distal end, the navigation method uses a deep network reinforcement learning method (DQN, standing for “Deep Q-Learning”, or DDPG, standing for “Deep Deterministic Policy Gradient”), said method having two drawbacks that are first of all the need for prior training and next the difficulty of generalizing to any type of artery topology and geometry of a patient.

In the whole of the prior art considered, the invention notes that there does not exist any automatic navigation system capable of all the following:
being effective for a wider family of elongate flexible medical instrument, and in particular one where the distal end is free, i.e. is not steerable (unlike for example endoscopes, the tip of which is often steerable, in particular by several cables attached to this tip),
being easily usable and applicable to a wide range of blood circulatory systems for numerous patients, or even to a wide range of portions of blood circulatory systems of a patient,
providing effective, rapid and secure slaving to an optimized path, and this within a blood circulatory system of a patient, which is intrinsically imbricated, vast and complex.

### OBJECTS OF THE INVENTION

The aim of the present invention is to provide a catheter robot, a navigation system and a navigation method at least partially overcoming the aforementioned drawbacks.

More particularly, the invention aims to provide, to do this, a navigation system for an elongate flexible medical instrument of a catheter robot implementing an automatic navigation method, as well as the associated catheter robot and the associated navigation method, which implements:
a special modeling, adapted to elongate flexible medical instruments without a steerable distal end, based on a meticulous monitoring of the movement of this non-steerable free distal end, in particular during its potential interaction with a blood vessel wall in the blood circulatory system of the patient,
   so as to obtain an automatic navigation that is efficient (rapid short travel in the blood circulatory system) and secure (without risk of passing through the wall of a blood vessel),
planning of the sequence of movement commands for the elongate flexible medical instrument that provides effective following of a given path in an optimized manner in the cleverly modeled system, based on a method that has proved particularly simple and effective for this type of travel in a vast system with multiple branches, diversions and re-crossings, which is the tree search,
   so as to obtain an automatic navigation that is efficient (rapid short travel in the blood circulatory system) and stable (no continuous or excessively frequent need for error correction, or even complete recalculation for reasons of divergence).

According to the invention, a catheter robot is provided comprising an automatic navigation system, for an elongate flexible medical instrument that is a catheter or a catheter guide, with free distal end, implementing an automatic navigation method successively comprising: a step of creating a modeling: of the elongate flexible medical instrument, of a blood circulatory system of a patient, along which the elongate flexible medical instrument is intended to move, of the interaction between this system and this elongate flexible medical instrument, by modeling of the contact between this elongate flexible medical instrument and one or more blood vessel walls in this system, from one or more images of the actual blood circulatory system of this patient, a step of determining: the position of a start point in the modeled system, the position of a point of arrival in the modeled system, a path to be followed, by the elongate flexible medical instrument, between the start point and the point of arrival, by determining a path, along the system modeled, between the start point and the point of arrival, with preferentially obligatory points of passage in the modeled system, a step of planning a sequence of movement commands for the elongate flexible instrument, obtained by a tree search procedure which, by using the modeling of the elongate flexible medical instrument and the modeling of said contact: simulates various possible movements of the elongate flexible medical instrument in the system modeled, evaluates the results of the simulation of these various possible movements, selects the most promising simulated movements among these various possible movements, to best follow the determined path with respect to one or more given criteria, a step of implementing the planned sequence of commands, along the actual blood circulatory system of said patient, with compensation for any differences with respect to the given path along the modeled system, by closed-loop regulation.

A catheter with a free distal end is a catheter with a distal end that is not steerable in a given direction. When the distal end of this catheter arrives at a branch in the blood circulatory system, this distal end cannot be deformed by an actuator to be directed on one side to go into the selected branch at the branching, but can only be turned about its longitudinal axis, which is also its progression axis. The free distal end of the catheter is also called the tip of the catheter. A catheter with a free distal end has no mechanism for deforming its distal end.

A catheter guide with a free distal end is a catheter guide with a distal end that is not steerable in a given direction. When the distal end of this catheter guide arrives at a branching in a blood circulatory system, this distal end cannot be deformed by an actuator to be directed to one side to go into the selected branch at the branching, but can only be turned about its longitudinal axis, which is also its progression axis. The free distal end of the catheter guide is also referred to as the catheter guide tip. A catheter guide with a free distal end has no mechanism for deforming its distal end.

Conversely, a steerable catheter is a catheter the distal end of which can be controlled by the practitioner so that he directs the distal end of the catheter in the required manner. The practitioner controls the catheter by deforming the distal end via a deformation mechanism, for example cables that are pulled or released according to the required deformation.

The images of the actual blood circulatory system of this patient can include or be for example radios, or X-ray images, and in particular be two-dimensional (2D) images. The images of the actual blood circulatory system of the patient may also include a 3D Coroscan (standing for “Coronary Computed Tomography Scan”), and/or an MRI. The images of the actual blood circulatory system of the patient may include three-dimensional (3D) images. The three-dimensional images may be constructed from two-dimensional images.

According to the invention, a catheter robot is also provided, comprising an automatic navigation system, for an elongate flexible medical instrument that is a catheter or a catheter guide, with free distal end, implementing an automatic navigation method comprising successively: a step of creating a modeling: of the elongate flexible medical instrument, of a blood circulatory system of a patient, along which the elongate flexible medical instrument is intended to move, of the interaction between this system and this elongate flexible medical instrument, by modeling of the contact between this elongate flexible medical instrument and one or more blood vessel walls in this system, from one or more images of the actual blood circulatory system of this patient, a step of determining: the position of a start point in the modeled system, the position of a point of arrival in the modeled system, a path to be followed, by the elongate flexible medical instrument, between the start point and the point of arrival, by determining a path, along the system modeled, between the start point and the point of arrival, with preferentially obligatory points of passage in the modeled system, a step of planning a sequence of movement commands for the elongate flexible instrument, obtained by a tree search procedure which, by using the modeling of the elongate flexible medical instrument and the modeling of said contact: simulates various possible movements of the elongate flexible medical instrument in the system modeled, evaluates the results of the simulation of these various possible movements, selects the most promising simulated movements among these various possible movements, to best follow the determined path with respect to one or more given criteria.

According to the invention, an automatic navigation system is also provided, for an elongate flexible medical instrument that is a catheter or a catheter guide, of a catheter robot, with free distal end, implementing an automatic navigation method successively comprising: a step of creating a modeling: of the elongate flexible medical instrument, of a blood circulatory system of a patient, along which the elongate flexible medical instrument is intended to move, of the interaction between this system and this elongate flexible medical instrument, by modeling of the contact between this elongate flexible medical instrument and one or more blood vessel walls in this system, from one or more images of the actual blood circulatory system of this patient, a step of determining: the position of a start point in the modeled system, the position of a point of arrival in the modeled system, a path to be followed, by the elongate flexible medical instrument, between the start point and the point of arrival, by determining a path, along the system modeled, between the start point and the point of arrival, with preferentially obligatory points of passage in the modeled system, a step of planning a sequence of movement commands for the elongate flexible medical instrument, obtained by a tree search procedure which, by using the modeling of the elongate flexible medical instrument and the modeling of said contact: simulates various possible movements of the elongate flexible medical instrument in the system modeled, evaluates the results of the simulation of these various possible movements, selects the most promising simulating movements among these various possible movements, to best follow the determined path with respect to one or more given criteria, a step of implementing the planned sequence of commands, along the actual blood circulatory system of said patient, with compensation for any differences with respect to the given path along the modeled system, by closed-loop regulation.

According to the invention, an automatic navigation system is next provided, for an elongate flexible medical instrument that is a catheter or a catheter guide, of a catheter robot, with free distal end, implementing an automatic navigation method successively comprising: a step of creating a modeling: of the elongate flexible medical instrument, of a blood circulatory system of a patient, along which the elongate flexible medical instrument is intended to move, of the interaction between this system and this elongate flexible medical instrument, by modeling of the contact between this elongate flexible medical instrument and one or more blood vessel walls in this system, from one or more images of the actual blood circulatory system of this patient, a step of determining: the position of a start point in the modeled system, the position of a point of arrival in the modeled system, a path to be followed, by the elongate flexible medical instrument, between the start point and the point of arrival, by determining a path, along the system modeled, between the start point and the point of arrival, with preferentially obligatory points of passage in the modeled system, a step of planning a sequence of movement commands for the elongate flexible instrument, obtained by a tree search procedure which, by using the modeling of the elongate flexible medical instrument and the modeling of said contact: simulates various possible movements of the elongate flexible medical instrument in the system modeled, evaluates the results of the simulation of these various possible movements, selects the most promising simulated movements among these various possible movements, to best follow the determined path with respect to one or more given criteria.

According to the invention, an automatic navigation method is also provided, for an elongate flexible medical instrument that is a catheter or a catheter guide, with free distal end, of a catheter robot, comprising successively: a step of creating a modeling: of the elongate flexible medical instrument, of a blood circulatory system of a patient, along which the elongate flexible medical instrument is intended to move, of the interaction between this system and this elongate flexible medical instrument, by modeling of the contact between this elongate flexible medical instrument and one or more blood vessel walls in this system, from one or more images of the actual blood circulatory system of this patient, a step of determining: the position of a start point in the modeled system, the position of a point of arrival in the modeled system, a path to be followed, by the elongate flexible medical instrument, between the start point and the point of arrival, by determining a path, along the system modeled, between the start point and the point of arrival, with preferentially obligatory points of passage in the modeled system, a step of planning a sequence of movement commands for the elongate flexible medical instrument, obtained by a tree search procedure which, by using the modeling of the elongate flexible medical instrument and the modeling of said contact: simulates various possible movements of the elongate flexible medical instrument in the system modeled, evaluates the results of the simulation of these various possible movements, selects the most promising simulated movements among these various possible movements, to best follow the determined path with respect to one or more given criteria, a step of implementing the planned sequence of commands, along the actual blood circulatory system of said patient, with compensation for any differences with respect to the given path along the modeled system, by closed-loop regulation.

According to the invention, finally, an automatic navigation method is provided, for an elongate flexible medical instrument that is a catheter or a catheter guide, with free distal end, of a catheter robot, comprising successively: a step of creating a modeling: of the elongate flexible medical instrument, of a blood circulatory system of a patient, along which the elongate flexible medical instrument is intended to move, of the interaction between this system and this elongate flexible medical instrument, by modeling of the contact between this elongate flexible medical instrument and one or more blood vessel walls in this system, from one or more images of the actual blood circulatory system of this patient, a step of determining: the position of a start point in the modeled system, the position of a point of arrival in the modeled system, a path to be followed, by the elongate flexible medical instrument, between the start point and the point of arrival, by determining a path, along the system modeled, between the start point and the point of arrival, with preferentially obligatory points of passage in the modeled system, a step of planning a sequence of movement commands for the elongate flexible medical instrument, obtained by a tree search procedure which, by using the modeling of the elongate flexible medical instrument and the modeling of said contact: simulates various possible movements of the elongate flexible medical instrument in the system modeled, evaluates the results of the simulation of these various possible movements, selects the most promising simulated movements among these various possible movements, to best follow the determined path with respect to one or more given criteria.

According to preferred embodiments, the invention comprises one or more of the following features, which can be used separately or in partial combination with each other or in complete combination with each other, with any one of the aforementioned objects of the invention.

Preferably, said automatic navigation method is able to be implemented in real time, said tree search procedure of the planning step simultaneously implements in parallel, for several possible different movements of the elongate flexible medical instrument in the modeled system, for at least two or at least three or at least four possible different movements, the following planning cycle: simulating a possible movement of the elongate flexible medical instrument in the modeled system, evaluating the result of the simulation of this possible movement, selecting the simulated movement if it is one of the most promising ones among the various possible movements, to best follow the determined path with respect to one or more given criteria.

Thus not only will the automatic navigation used be effective, in that a path determined in an optimized manner will be followed as close by as possible, but also this travel will be implemented in a time that is appreciably shorter than expected, by virtue of the parallelization of the travel over the tree, which certainly will give rise to a little additional work but will save an appreciable and particularly precious amount of time for work on a patient to be carried out in real time. The tree search procedure lends itself particularly well to this parallelization of the travel over the tree, which is not necessarily the case with a certain number of other techniques for determining and travelling over an optimized path.

Preferably, if at the end of the implementation step, one or more differences to be compensated for are assessed as being too great, compared with one or more given criteria: then an adjustment of the modeling is made, and on the adjusted modeling the following are next implemented: first a new step of determining a new path, next a new step of planning a new sequence of commands for movement of the elongate flexible medical instrument, from the new path determined, and finally, where applicable, a new step of implementing the new planned sequence of commands.

Thus in the case either of a particularly long path or of travel in a particularly tangled portion of the blood circulatory system of the patient, this combination of an adjustment of the modeling and an iteration of some of the steps of progress with the automatic navigation, will ensure:
firstly, convergent and close guidance along the determined path,
and secondly appreciably limiting the quantity of computing and simulation to be implemented, by making it possible to use simplifications in the simulation or in the exploration of the tree search algorithm.

Thus a good compromise can be achieved between relative simplicity of the model used and a relatively low frequency of adjustments, while reducing or even eliminating the risk of having the system diverge (for reasons of excessively great differences between reality and its modeling).

Preferably, said modeling creation step implements a representation of the physical system encompassing both the elongate flexible medical instrument and the blood circulatory system of a patient, along which the elongate flexible medical instrument is intended to move, by finite element simulation.

Thus this type of method operates particularly well for the type of physical system to be modeled, and in particular the type of numerous small successive movements of the elongate flexible medical instrument in the blood circulatory system of the patient.

Preferentially, the elongate flexible medical instrument is modeled by Kirchhoff beams or by Cosserat beams.

Thus this type of method operates particularly well for modeling an elongate flexible medical instrument that is as long, flexible and fine as a catheter or a catheter guide.

Preferably, the blood circulatory system of the patient is modeled: either by point clouds or by meshings, or by center lines associated with their respective diameters, or by implicit surfaces.

Thus this type of method operates particularly well for modeling blood vessels in a blood circulatory system of a patient.

Preferably, the modeling of the blood circulatory system of the patient incorporates the movements affecting this blood circulatory system of the patient, and preferably incorporates the deformations of the heart of the patient when it beats and/or the deformations caused by the breathing of the patient.

Thus the automatic navigation implemented will be dynamic, and no longer only static, by integrating the changes in the geometry of the blood circulatory system of the patient.

Preferably, in said determination step: said start point in the modeled system is positioned at the outlet of the guide catheter of the catheter robot, at the ostium, and said point of arrival in the modeled system is positioned in the patient, at the lesion to be treated.

Thus the elongate flexible medical instrument can easily be guided truly throughout the path that it will travel.

Preferably, in said determination step: the path to be followed is determined by interpolation between said start point and said point of arrival, and preferentially by use of a Dijkstra graph travel algorithm applied to the center lines of the blood vessels of the blood circulatory system.

Thus this type of method operates particularly well for determining a path in a particularly large and complex system of possible pathways, as may be a patient's blood circulatory system.

Preferably, said tree search procedure evaluates the results of the simulation of these various possible movements by attributing marks to each branch of the tree, and then calculating a value for each node of the tree from the marks attributed to the branches leading to this node, selects the most promising simulated movements among these various possible movements that are the simulated movements leading to the node with the highest value, to guide its exploration in the tree.

Thus following an optimized path in a shortened time will be further improved.

Preferably, said tree search procedure attributes the marks in the same way: if the elongate flexible medical instrument advances along the determined path, then the corresponding possible movement of the elongate flexible medical instrument in the modeled system receives a positive mark, if the elongate flexible medical instrument advantages other than along the determined path or if the elongate flexible medical instrument retracts or if the elongate flexible medical instrument stagnates along the determined path, then the corresponding possible movement of the elongate flexible medical instrument in the modeled system receives a zero or negative mark.

Preferably, said tree search procedure attributes the marks in the following way: if the possible movement of the elongate flexible medical instrument along the determined path is considered to be similar to that which would result from the action of a doctor selected as a reference, then this possible movement receives a positive mark, if the possible movement of the elongate flexible medical instrument along the determined path is considered to be different from that which would result from the action of a doctor selected as a reference, then this possible movement receives either a zero mark or a negative mark.

Preferably, said tree search procedure partly attributes the marks in the following first manner: if the elongate flexible medical instrument advances along the determined path, then the corresponding possible movement of the elongate flexible medical instrument in the modeled system receives a positive mark, if the elongate flexible medical instrument advances otherwise than along the determined path or if the elongate flexible medical instrument retracts or if the elongate flexible medical instrument stagnates along the determined path, then the corresponding possible movement of the elongate flexible medical instrument in the modeled system receives a zero or negative mark, also attributes, partly, the marks in the following second manner: if the possible movement of the elongate flexible medical instrument along the determined path is considered to be similar to that which would result from the action of a doctor selected as a reference, then this possible movement receives a positive mark, if the possible movement of the elongate flexible medical instrument along the determined path is considered to be different from that which would result from the action of a doctor selected as a reference, then this possible movement receives either a zero mark or a negative mark, uses, with preferentially predetermined weightings, optionally different from each other, respectively the first manner and the second manner.

Thus the richness of the criteria for evaluating the possible movements for the elongate flexible medical instrument will improve the probability of having an even more optimized path and to travel it even more quickly.

Preferably, the value of each node is calculated by totaling the marks of each branch leading to this node, or the value of each node is calculated according to the UCB method (UCB means “Upper Confidence Bound”).

Preferably, said tree search procedure returns to pass through a node with a less good value, after a node with a better value has proved to be a dead end. A node that proves to be a dead end is a node which, promising at the start, does not live up to its promises and it is in fact not advantageous for the elongate flexible medical instrument to follow the corresponding path.

Thus the guidance is made even more convergent and tight along the determined path, without risk of remaining blocked and then being obliged to reinitiate a new path calculation that would have been able to be avoided.

Preferentially, said tree search procedure is an algorithm:
either a Monte Carlo tree search (MCTS),
or upper bound of the confidence interval applied to the trees (UCT, standing for “Upper Confidence Bound applied to Trees”),
or optimized planner for deterministic systems (OPD),
or open loop optimized planner (OLOP).

Preferably, said step of implementing the planned command sequence is implemented by direct command effecting the movements of the actuators controlling the movements of the elongate flexible medical instrument.

Thus the command is more simply calculated.

Preferably, said step of implementing the planned command sequence is implemented by inverse command calculating the movements of the actuators from the movements of the elongate flexible medical instrument.

Thus the command is more precisely calculated.

Preferentially, the blood vessels of the blood circulatory system are arteries of the blood circulatory system. These blood vessels could however be veins, or arteries and veins.

Preferably, the catheter robot comprising an automatic navigation system, for several elongate flexible medical instruments among which there is at least one catheter and a catheter guide associated with this catheter, these elongate flexible medical instruments being with a free distal end.

Thus the guidance implemented by this automatic navigation is more complete.

The catheter may be a so-called “stent” or “balloon” catheter that is hollow. The catheter guide may be a metal wire that is solid.

Other features and advantages of the invention will emerge from the reading of the following description of a preferred embodiment of the invention, given by way of example and with reference to the accompanying drawings.

### BRIEF DESCRIPTION OF THE DRAWINGS

FIG. 1 shows schematically an example of a system including a catheter robot with a patient installed in a medical imaging device.

FIG. 2 shows schematically an example of simulation of the navigation of a catheter guide in a model of coronary arteries of a patient, in an automatic navigation method according to an embodiment of the invention.

FIG. 3 shows schematically an example of a representation of 5 nodes of the tree connected by the 4 possible actions, in an automatic navigation method according to an embodiment of the invention.

FIG. 4 shows schematically an example of a first step of a first type of use of a tree search algorithm, in an automatic navigation method according to an embodiment of the invention.

FIG. 5 shows schematically an example of a second step of a first type of use of a tree search algorithm, in an automatic navigation method according to an embodiment of the invention.

FIG. 6 shows schematically an example of a third step of a first type of use of a tree search algorithm, in an automatic navigation method according to an embodiment of the invention.

FIG. 7 shows schematically an example of a fourth step of a first type of use of a tree search algorithm, in an automatic navigation method according to an embodiment of the invention.

FIG. 8 shows schematically an example of a fifth step of a first type of use of a tree search algorithm, in an automatic navigation method according to an embodiment of the invention.

FIG. 9 shows schematically an example of a sixth step of a first type of use of a tree search algorithm, in an automatic navigation method according to an embodiment of the invention.

FIG. 10 shows schematically an example of a seventh step of a first type of use of a tree search algorithm, in an automatic navigation method according to an embodiment of the invention.

FIG. 11 shows schematically an example of an eighth step of a first type of use of a tree search algorithm, in an automatic navigation method according to an embodiment of the invention.

FIG. 12 shows schematically an example of a ninth step of a first type of use of a tree search algorithm, in an automatic navigation method according to an embodiment of the invention.

FIG. 13 shows schematically an example of a tenth step of a first type of use of a tree search algorithm, in an automatic navigation method according to an embodiment of the invention.

FIG. 14 shows schematically an example of an eleventh step of a first type of use of a tree search algorithm, in an automatic navigation method according to an embodiment of the invention.

FIG. 15 shows schematically an example of a twelfth step of a first type of use of a tree search algorithm, in an automatic navigation method according to an embodiment of the invention.

FIG. 16 shows schematically an example of a step of a second type of use of a tree search algorithm, in an automatic navigation method according to an embodiment of the invention.

FIG. 17 shows schematically an example of a step that follows the step of FIG. 16.

FIG. 18 shows schematically an example of a step that follows the step of FIG. 17.

FIG. 19 shows schematically an example of a first step of a third type of use of a tree search algorithm, in an automatic navigation method according to an embodiment of the invention.

FIG. 20 shows schematically an example of a second step of a third type of use of a tree search algorithm, in an automatic navigation method according to an embodiment of the invention.

FIG. 21 shows schematically an example of a third step of a third type of use of a tree search algorithm, in an automatic navigation method according to an embodiment of the invention.

FIG. 22 shows schematically an example of a fourth step of a third type of use of a tree search algorithm, in an automatic navigation method according to an embodiment of the invention.

FIG. 23 shows schematically an example of a fifth step of a third type of use of a tree search algorithm, in an automatic navigation method according to an embodiment of the invention.

FIG. 24 shows schematically an example of a sixth step of a third type of use of a tree search algorithm, in an automatic navigation method according to an embodiment of the invention.

FIG. 25 shows schematically an example of a seventh step of a third type of use of a tree search algorithm, in an automatic navigation method according to an embodiment of the invention.

FIG. 26 shows schematically an example of an eighth step of a third type of use of a tree search algorithm, in an automatic navigation method according to an embodiment of the invention.

FIG. 27 shows schematically an example of a ninth step of a third type of use of a tree search algorithm, in an automatic navigation method according to an embodiment of the invention . . .

### DETAILED DESCRIPTION OF EMBODIMENTS OF THE INVENTION

The invention proposes an automation of the navigation of one or more flexible elongate medical instruments, for example a catheter guide and a catheter. This automation will successively implement the following steps. First a first step of creating a model of the elongate flexible medical instruments, for example guide, catheter, catheter guide, and the environment thereof, for example the arteries and/or the veins of the blood system of the patient wherein the elongate flexible medical instruments will move. Next, a second step of defining a start point, for example the ostium, and a point of arrival, for example a distal part of the artery close to the lesion to be treated in the patient, in the model generated at the previous step, so as to obtain a path to be followed for the elongate flexible medical instrument in the blood system of the patient, for example following a path along the arteries of the patient. Then a third step of planning a sequence of commands to be implemented so that the elongate flexible medical instrument follows the previously defined path, this planning being implemented by a tree search of the various movements of the elongate flexible medical instrument, this planning being implemented in the previously generated model. Finally, a fourth step of implementing the sequence of previously defined commands on the catheter robot.

In the first step, the model that will be constructed during this first step is a representation of the physical system comprising the arteries of the blood system of the patient, obtained from medical imaging of the patient, and the elongate flexible medical instruments that navigate in these arteries of the blood system of the patient. Several methods can be used for generating this model.

In one variant used, the model is a finite element simulator, of the simulation FEM type. The arteries of the blood system of the patient are represented by a 3D geometric model (three-dimensional) such as for example point cloud, meshing, center line and diameter, implicit surface. The elongate flexible medical instruments are modeled by beams, for example by Kirchhoff beams or by Cosserat beams.

The model can incorporate the movements of the environment, for example the deformations of the heart when it beats.

In the second step, the start point and the point of arrival are 3D coordinates defined in the previously generated model. The start point corresponds in general to the outlet of the catheter guide at the ostium. The point of arrival in general corresponds to the lesion to be treated, or more precisely to a selected point in the lesion to be treated.

The path is obtained by interpolation between the start point and the point of arrival. The path may for example be determined by a Dijkstra graph travel algorithm applied to the center line of the arteries of the blood system of the patient.

In the third step, the planning of the sequence of commands is obtained by tree search, which is also referred to as predictive command (or “Model Predictive Control”).

This tree search is implemented by exploring sequences of commands in the model to predict the effects thereof, and thus to find a suitable command sequence.

The algorithm may for example simulate each possible movement from a start position, and each of the states of the model, a state of the model including the position of the elongate flexible medical instrument as well as the position of the arteries of the blood system of the patient, resulting from these commands, are marked in order to explore as a priority the states with the best mark. The mark given by the algorithm may also be termed recompense.

The algorithm may for example interact with its environment by 4 discrete actions, which are:
either advancing by N mm (millimeters),
or turning through M radians,
or turning through-M radians,
or retracting by N mm,
N and M advantageously being predefined constants.

The various states of the physical system constitute the nodes of a tree to be travelled over. The branches of this tree are the actions making it possible to pass from one node to the other, and therefore from one state of the physical system to the other.

Each node of the tree is associated with a value evaluating the potential of each node so that a node with the highest value has more chances of being on the optimal path between the start point and the point of arrival than a node with a lower value. This value is preferably determined in the following manner:

Vnode=Vparent+g^{d-1}*Recompense+(g^{d}/(1−g))

With Vnode being the value of the current node, Vparent the value of the parent node, g the discount factor, d the depth of the node in the tree, Recompense the recompense corresponding to the action going from the parent node to the current node.

In a first possible variant of the third step, these marks are determined in the following manner. If the elongate flexible medical instrument advances along the path defined at the second step, then it receives a positive mark. In all the other cases, i.e. if the elongate flexible medical instrument advances in an arterial branch not corresponding to the path defined at the second step, or if the elongate flexible medical instrument retracts, then it receives a zero or negative mark.

In a second possible variant of the third step, the marks (or recompenses) are attributed so that the movements that are closest to those that would be performed by a doctor obtain the best marks (“Inverse Reinforcement Learning”), and the movements that would not be selected by a doctor obtain zero or negative marks.

The algorithm attributes a value based on the marks to each node in the tree and uses this value for guiding its exploration, the exploration being performed as a priority on the nodes with the highest value.

According to a first possible option, this value can be the total recompense of the node and of all its parents in the tree. According to a second possible option, the value is determined according to the UCB method (standing for “Upper Confidence Bound”).

If a branch that appeared promising at the start is finally a “dead end”, there is a rearward movement towards the branches which, initially, had a less high value, and therefore less promising at the start and which therefore had not been selected at that moment.

In a third possible variant of the third step, the exploration of the movement by the tree search is implemented not systematically, but with a predetermined algorithm, for example resulting from an end-to-end learning. The exploration of the movements by the tree search can also be implemented by imitating movements that would have been made by a doctor. In this third variant, the exploration of the movements is implemented during A % of the time by using the values determined as at the first variant, and B % of the time by attempting to imitate the movements of a doctor as at the second variant. The third variant amounts for example to combining for 50% of the time the first variant and for 50% of the time the second variant, or for example combining for 80% of the time the first variant and for 20% of the time the second variant.

Examples of a tree search algorithm are:
either a Monte Carlo tree search (MCTS),
or upper bound of the confidence interval applied to the trees (UCT, standing for “Upper Confidence Bound applied to Trees”),
or optimized planner for deterministic systems (OPD),
or open loop optimistic planning (OLOP).

In the tree search algorithm used here, the tree is developed in 3 phases:
First of all a selection, where the node with the highest value is selected,
Then an expansion, where all the possible actions from this selected node are simulated and new child nodes are created,
Next a backpropagation, where the values of the newly created child nodes are calculated from the recompenses associated with each action. These values are next propagated first to the parent nodes and next recursively to the root node,
These 3 steps can be repeated several times. Advantageously the number of repetitions of these 3 steps is predefined, thus making it possible to ensure that the algorithm converges in a finite time. This number of repetitions is called the budget.

In a fourth step, the sequence of commands planned at the third step is implemented by the catheter robot in order to move the elongate flexible medical instrument. This implementation can be done by a direct command algorithm, wherein the command is a movement of the actuators, or an inverse command algorithm in which the command is a movement of the elongate flexible medical instrument from which the corresponding movements of the actuators are calculated.

A closed-loop control system is used for implementing the sequence of commands. This closed loop uses the medical imaging system in order to follow over time the position of the elongate flexible medical instrument that is being moved by the catheter robot, and also in order to check that the elongate flexible medical instrument is indeed following the path defined during the second step. If differences in position are noted, then there are two possibilities that are either being able to compensate for these differences with the closed-loop regulation or, if these differences are too great, to proceed with a replanning of the command sequence from the current state of the system, which becomes the new start point for the second step, and said second step is then reiterated.

The use of the closed loop with the medical imaging can make it possible no to plan all the commands to go as far as the point of arrival, but to plan only the commands over a certain step. It is in fact considered in this case that, in particular because of the simplifications of the model, after several time steps, the model would not entirely correspond to reality, and that then it could be more advantageous to implement an adjustment with the medical imagery and then replanning.

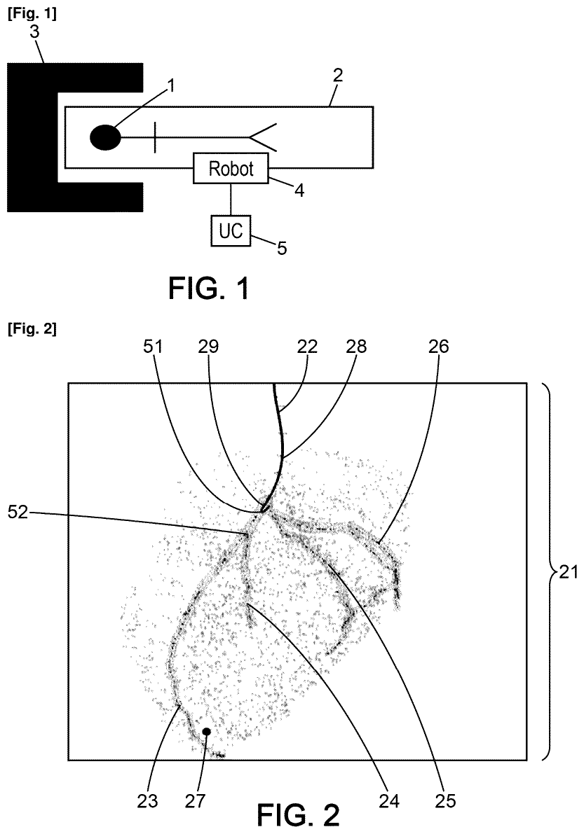

FIG. 1 shows schematically an example of a system including a catheter robot with a patient installed in a medical imaging device.

The patient 1 is for example lying on an examination bed or table 2, to which a catheter robot 4 controlled by a control unit 5 is attached, using the images of the patient 1 that are obtained by a medical imaging system 3.

FIG. 2 shows schematically an example of simulation of the navigation of a catheter guide in a model of coronary arteries of a patient, in an automatic navigation method according to one embodiment of the invention.

The blood system 21 of the patient comprises a main artery 22 that will gradually divide into several secondary arteries 23, 24, 25, 26. The point of arrival, i.e. the lesion of the patient to be treated, is the point 27 located along the secondary artery 23. The catheter guide 28 has a curved distal end 29 that is located on the main artery 22 at a first branching 51. At this first branching 51, the curved distal end 29 of the catheter guide 28, which descends (on FIG. 2) along the main artery 22, will have to take the right-hand branch with respect to its direction of movement, towards the secondary arteries 23 and 24, and leave on one side the left-hand branch with respect to its direction of movement, which would go towards the secondary arteries 25 and 26. After having progressed again along the main artery 22, from the first branching 51, the curved distal end 29 of the catheter guide 28 will arrive at a second branching 52. At this second branching 52, the curved distal end 29 of the catheter guide 28, which continues to descend (on FIG. 2) along the main artery 22, will have to take the right-hand branch with respect to its direction of movement, towards the secondary artery 23, and leave on one side the left-hand branch with respect to its direction of movement, which would go towards the secondary artery 24. After having progressed again along the secondary artery 23, from the second branching 52, the curved distal end 29 of the catheter guide 28 will arrive at its point of arrival 27 in the lesion of the patient to be treated.

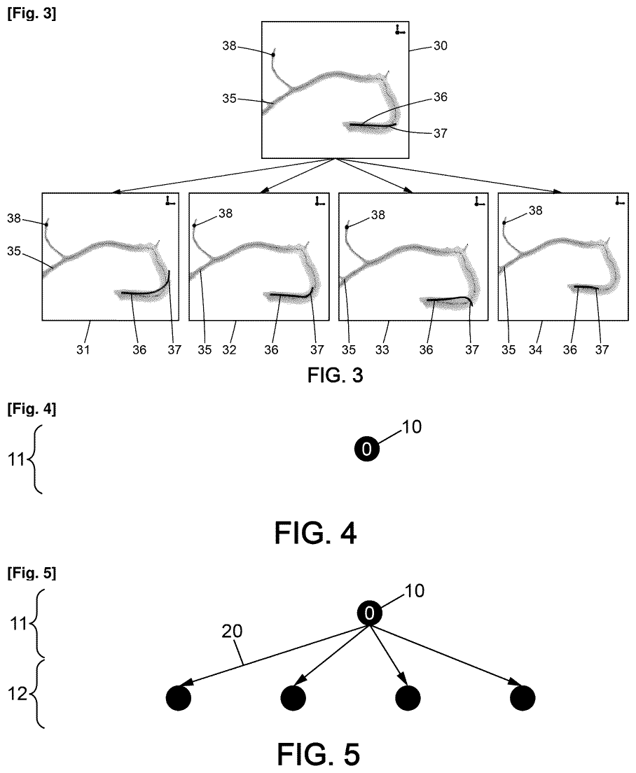

FIG. 3 shows schematically an example of representation of 5 nodes of the tree connected by the 4 possible actions, in an automatic navigation method according to an embodiment of the invention.

From the position 30, on which the arteries 35 of the blood system of the patient can be seen, and the curved distal end 37 of a catheter guide 36 that progresses along these arteries 35, towards its point of arrival 38, which is located in the lesion of the patient to be treated. From this position 30, 4 actions are possible for the curved distal end 37 of a catheter guide 36:
either advancing along these arteries 35, which is shown in the position 31, where the curved distal end 37 of a catheter guide 36 that has advanced towards its point of arrival 38 can be seen,
or turning in a direction about its longitudinal axis, which is shown in the position 32, where the curved distal end 37 of a catheter guide 36 can be seen, that has remained in place with respect to its point of arrival 38, but which has turned about the longitudinal axis of the catheter guide 36, in one direction, for example in the clockwise direction, for example to better approach the next branching or the next artery curvature 35, or to better advance thereafter,
or turning in the other direction about its longitudinal axis, which is shown in the position 33, where the curved distal end 37 of a catheter guide 36 can be seen, which has remained in position with respect to its point of arrival 38, but which has turned about the longitudinal axis of the catheter guide 36, in the other direction, for example in the anticlockwise direction, for example to better approach the next branching or the next artery curvature 35, or to better retract thereafter,
or retracting along these arteries 35, which is shown in the position 34, where the curved distal end 37 of a catheter guide 36 that has retracted by moving away from its point of arrival 38 can be seen.

FIGS. 4 to 27 present a tree search algorithm that is simplified, and the purpose thereof is to describe the steps of: selection, expansion and backpropagation. Three variants are presented: sequential exploration (FIGS. 4 to 15), parallel exploration based on one criterion (FIGS. 16 to 18), and parallel exploration based on several criteria (FIGS. 19 to 27). The rules of this tree search algorithm are:
Selection: Selecting the leaf node or nodes (node without child) with the highest value;
Expansion: Systematically opening all the children of the leaf node or nodes selected;
Backpropagation: The value of a childless node (leaf node) is the recompense received during the action leading from its parent to this node, the value of a node having children is the maximum value of the sub-tree the root of which is this node.

FIGS. 4 to 15 show schematically an example of the various steps of a first type of use of a tree search algorithm, in a method for automatic navigation of an elongate flexible medical instrument in a blood system of a patient according to an embodiment of the invention.

This first type of use corresponds to a sequential use of the tree search algorithm, the expansion of the tree takes place in three phases:
First a selection, where the leaf node with the highest value is selected,
Then an expansion, where all the possible actions from this selected node are simulated and new child nodes are created,
Next a backpropagation, where the recompenses associated with each action are recovered and propagated first to the parent nodes and next recursively as far as the root node,
These 3 steps can be repeated several times. Advantageously the number of repetitions of these three steps is predefined, thus making it possible to ensure that the algorithm converges in a finite time. This number of repetitions is called the budget.

FIG. 4 shows schematically an example of a first step of a first type of use of a tree search algorithm, in an automatic navigation method according to an embodiment of the invention.

The various nodes 10 are distributed in various levels, here only the root node 11 is shown. Its default value is 0. This node being the only one, it is necessarily this one that is selected.

FIG. 5 shows schematically an example of a second step of a first type of use of a tree search algorithm, in an automatic navigation method according to an embodiment of the invention.

The various nodes 10 are distributed in various levels, the root level 11, and the first filiation level 12 (also referred to as depth 1).

The passage between two nodes, with different filiations, and therefore from a filiation level of the parent type to a filiation level of the child type, for example from the root level 11 to the first filiation level 12, is an action 20 (to be selected among the 4 actions already presented, which are: advancing or turning in one direction or turning in the other direction or retracting).

From the root node, the expansion of possible actions 20 is implemented, resulting in 4 new child nodes at the first filiation level 11.

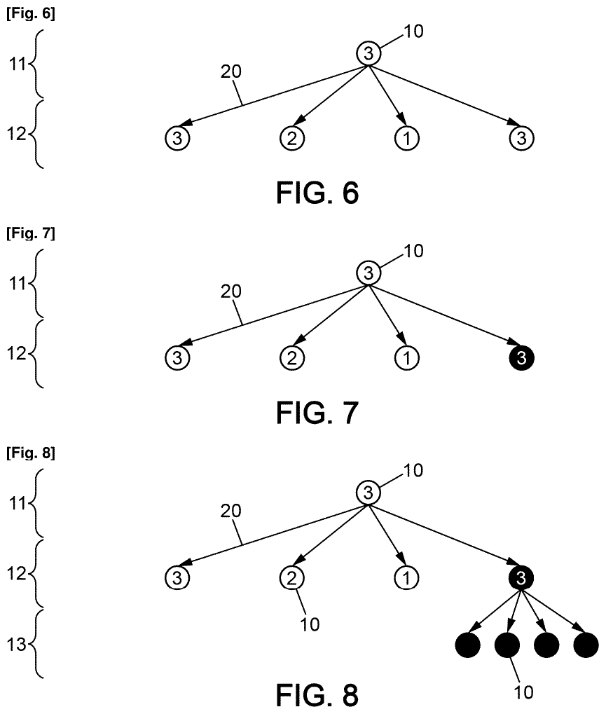

FIG. 6 shows schematically an example of a third step of a first type of use of a tree search algorithm, in an automatic navigation method according to an embodiment of the invention.

Once the values have been determined for these 4 new child nodes at the first filiation level 11, of this parent node, which is the root node, namely, from left to right, the values 3, 2, 1 and 3, the highest of these values, namely the value 3 of the first node or of the fourth node (starting from the left in FIG. 6) of the first filiation level 12, is backpropagated:
to its parent node, which is the root node, the value of which then changes from 0 to 3.

FIG. 7 shows schematically an example of a fourth step of a first type of use of a tree search algorithm, in an automatic navigation method according to an embodiment of the invention.

The various nodes 10 are distributed in various levels, the root level 11, the first filiation level 12 (also referred to as depth 1).

The “leaf” node (i.e. the nodes that do not have children in the tree) of the first filiation level 12 that is selected is the node furthest to the right on FIG. 7, with the highest value 3, since in fact the other leaf nodes have lower values (or equal ones, it would also have been possible to select the node furthest to the left of the first filiation level 12, which is also equal to 3), which are respectively 3, 2 and 1.

FIG. 8 shows schematically an example of a fifth step of a first type of use of a tree search algorithm, in an automatic navigation method according to an embodiment of the invention.

The various nodes 10 are distributed in various levels, the root level 11, the first filiation level 12, the second filiation level 13.

The passage between two nodes with different filiations, and therefore from a filiation level of the parent type to a filiation level of the child type, for example from the root level 11 to the first filiation level 12, or from the first filiation level 12 to the second filiation level 13, is an action 20.

From the leaf node selected at the first filiation level 12, the node furthest to the right of FIG. 8 with the highest value 3, the possible actions 20 are expanded, resulting in 4 new child nodes at the second filiation level 13.

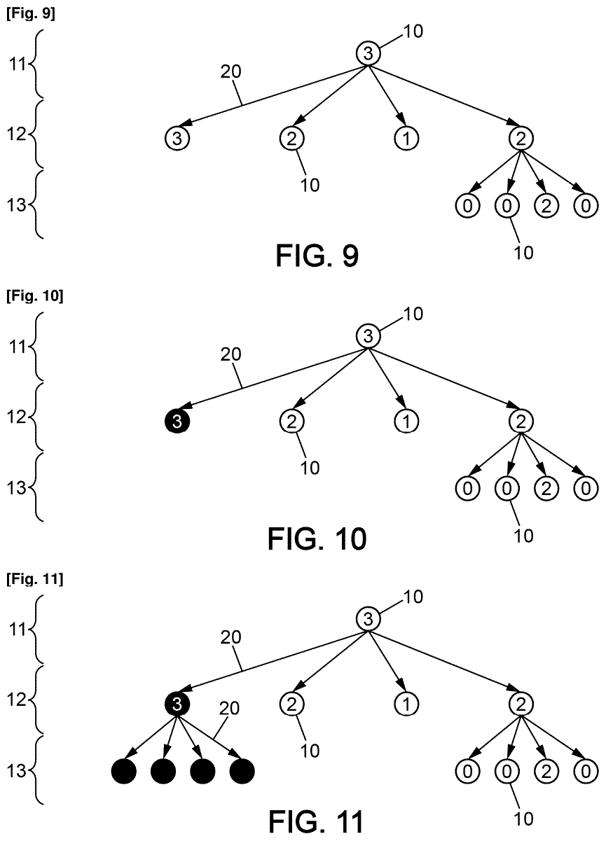

FIG. 9 shows schematically an example of a sixth step of a first type of use of a tree search algorithm, in an automatic navigation method according to an embodiment of the invention.

Once the values have been determined for these 4 new child nodes at the second filiation level 13 (namely from left to right the values 0, 0, 2 and 0), the highest of these values (namely the value 2 of the third node starting from the left in FIG. 9 of the second filiation level 13) is backpropagated:
to its parent node of the first filiation level 12, the value of which then changes from 3 to 2,
but not to the node of the root level 11, the value of which remains at 3, since the node furthest to the left still remains, of the first filiation level 12, of value 3.

FIG. 10 shows schematically an example of a seventh step of a first type of use of a tree search algorithm, in an automatic navigation method according to an embodiment of the invention.

The various nodes 10 are distributed in various levels, the root level 11, the first filiation level 12 (also referred to as depth 1) and the second filiation level 13 (also referred to as depth 2).

The leaf that is now selected is the node furthest to the left on FIG. 10, with the highest value 3 located in the first filiation level 12. This is because the other leaves have lower values that are respectively 2, 1, 0, 0, 2, 0.

FIG. 11 shows schematically an example of an eighth step of a first type of use of a tree search algorithm in an automatic navigation method according to an embodiment of the invention.

From the node selected at the first filiation level 12, the node furthest to the left on FIG. 11, with the highest value 3, the possible actions 20 are expanded, resulting in 4 new child nodes at the second filiation level 13, of this parent node previously selected at the first filiation level 12.

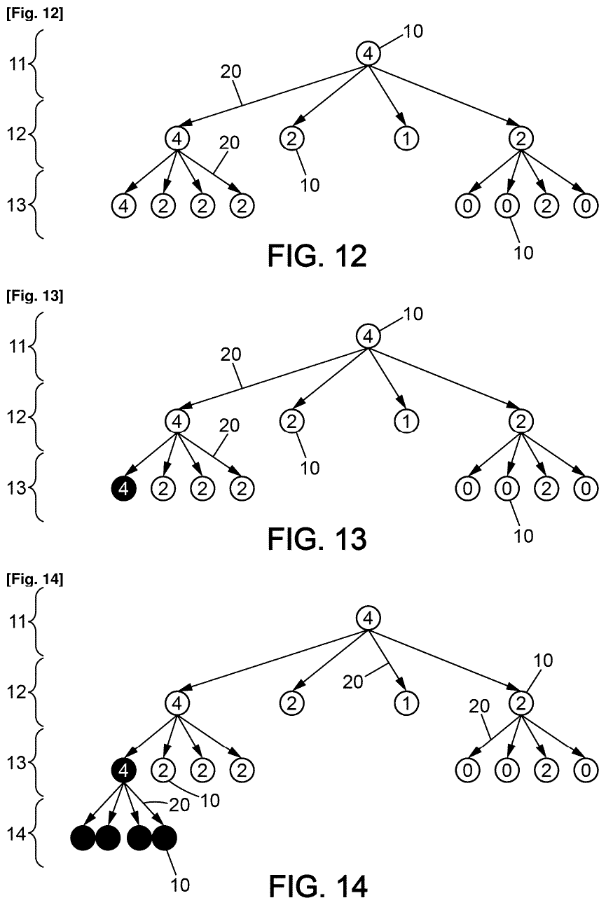

FIG. 12 shows schematically an example of a ninth step of a first type of use of a tree search algorithm, in an automatic navigation method according to an embodiment of the invention.

Once the values have been determined for these 4 new child nodes at the second filiation level 13, namely from left to right the values 4, 2, 2 and 2, the highest of these values (4) is backpropagated:
to its parent node of the first filiation level 12, the value of which then changes from 3 to 4,
as well as to the node of the root level 11, the value of which changes from 3 to 4.

FIG. 13 shows schematically an example of a tenth step of a first type of use of a tree search algorithm, in an automatic navigation method according to an embodiment of the invention.

The leaf node that is selected is the node of value 4 located at the second filiation level 13 and which is furthest to the left on FIG. 13. This is because the other leaf nodes have lower values, which are respectively 2, 2, 2, 2, 1, 0, 0, 2 and 0.

FIG. 14 shows schematically an example of an eleventh step of a first type of use of a tree search algorithm, in an automatic navigation method according to an embodiment of the invention.

From the leaf node selected at the second filiation level 13, the node furthest to the left on FIG. 14, with the highest value 4, the possible actions 20 are expanded, resulting in 4 new child nodes at the third filiation level 14.

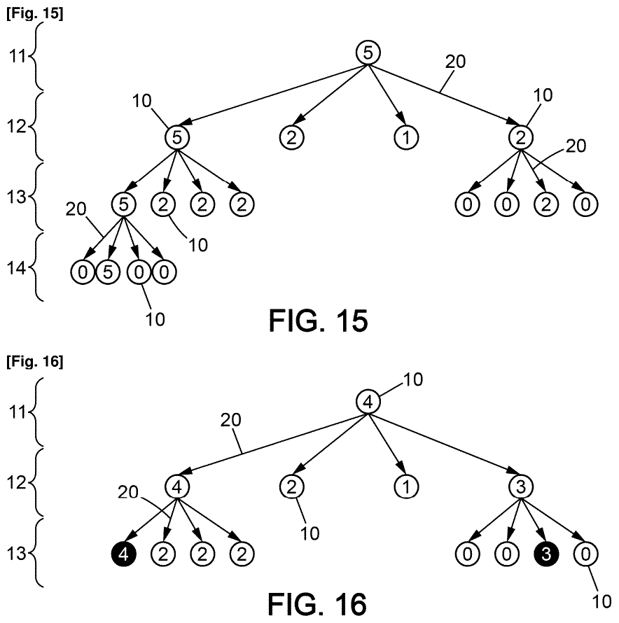

FIG. 15 shows schematically an example of a twelfth step of a first type of use of a tree search algorithm, in an automatic navigation method according to an embodiment of the invention.

Once the values have been determined for these 4 new child nodes at the third filiation level 14 (namely, from left to right, the values 0, 5, 0 and 0, the highest of these values (namely the value 5 of the second node starting from the left in FIG. 15 of the third filiation level 14) is backpropagated:
to its parent node of the second filiation level 13, the value of which then changes from 4 to 5,
and to its grandparent node of the first filiation level 12, the value of which changes from 4 to 5,
and to the node of the root level 11, the value of which changes from 4 to 5.

FIGS. 16 to 18 show schematically an example of the various steps of a second type of use of a tree search algorithm, in the context of the automatic navigation of an elongate flexible medical instrument in a blood system of a patient according to an embodiment of the invention. This second type of use corresponds to a parallel use based on the value of the leaf nodes. The operation is similar to the sequential operation, but instead of selecting only the leaf node with the highest value, several leaf nodes with the highest values are selected. Travelling over the tree in parallel makes it possible to explore more leaves in the same budget than in travelling over the tree sequentially. The value of the number of leaf nodes selected being related to the computing resources available. Here a selection of the 2 leaf nodes with the highest values will be considered, with an expansion of their 8 children, the recovery of the recompenses and the propagation of the value to the root of the tree.

FIG. 16 shows schematically an example of a step of a second type of use of a tree search algorithm, in an automatic navigation method according to an embodiment of the invention. The previous steps not being redescribed here for purposes of simplification. The steps that precede FIG. 16 may have been implemented in accordance with a sequential exploration, or a parallel exploration.

The two leaf nodes selected are: firstly the leaf node of value 4 that is located at the second filiation level 13 (the leaf node furthest to the left on FIG. 16 on the second filiation level 13); and secondly the leaf node of value 3, which is located at the second filiation level 13 (the leaf node furthest to the right on FIG. 16 at the second filiation level 13). This is because these two leaf nodes are the two leaf nodes with the highest values, the other leaf nodes having respectively the following values 2, 2, 2, 2, 1, 0, 0, 0 (from left to right).

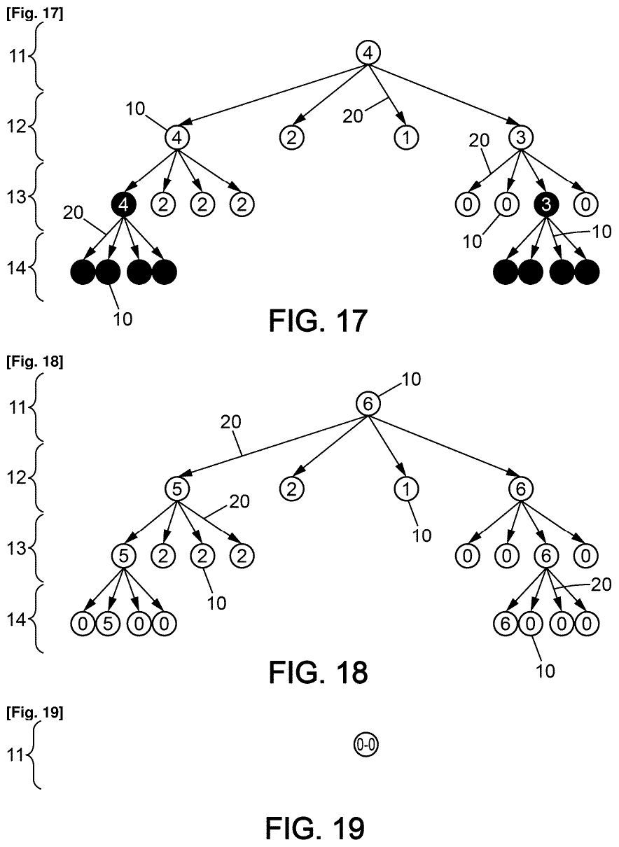

FIG. 17 shows schematically an example of a step of a second type of use of a tree search algorithm that follows the step illustrated on FIG. 16, in an automatic navigation method according to an embodiment of the invention.

From the first leaf node selected (of value 4), which is at the second filiation level 13, the possible actions 20 are expanded, resulting in 4 new child nodes at the third filiation level 14.

In parallel with this first expansion, from the second leaf node selected (or value 3), which is at the second filiation level 13, the possible actions 20 are expanded, resulting in 4 new child nodes at the third filiation level 14.

FIG. 18 shows schematically an example of a sixth step of a second type of use of a tree search algorithm that follows the step of FIG. 17, in an automatic navigation method according to an embodiment of the invention.

The values are determined for these 4 new child nodes at the third filiation level 14 of the first parent node previously selected at the second filiation level 13, the values being 0, 5, 0 and 0 (from left to right). Once the values have been determined for these 4 new child nodes at the third filiation level 14, the highest of these values (namely the value 5 of the second node starting from the left in FIG. 18 of the third filiation level 14) is backpropagated:
to its parent node of the second filiation level 13, the value of which then changes from 4 to 5,
and to its grandparent node of the first filiation level 12, the value of which changes from 4 to 5,
and to the node of the root level 11, the value of which should change from 4 to 5 (subject to the result of the other backpropagation).

The values are determined for these 4 new child nodes at the third filiation level 14 of the second parent node previously selected at the second filiation level 13, the values being 6, 0, 0 and 0 (from left to right). Once the values have been determined for these 4 new child nodes at the third filiation level 14, the highest of these values (namely the value 6 of the fifth node starting from the left in FIG. 18 of the third filiation level 14) is backpropagated:
to its parent node of the second filiation level 13, the value of which then changes from 3 to 6,
and to its grandparent node of the first filiation level 12, the value of which changes from 3 to 6,
and to the node of the root level 11, the value of which changes from 3 to 6 (this value 6 being higher than the previously backpropagated value (value 5)).

FIGS. 19 to 27 show schematically an example of the various steps of a third type of use of a tree search algorithm in the context of the automatic navigation of an elongate flexible medical instrument in a blood system of a patient according to an embodiment of the invention.

This third type of use corresponds to a parallel expansion based on a plurality of values (two values in the example presented on FIGS. 19-27). The operation is similar to the parallel operation described in FIGS. 16 to 18, but, instead of selecting the nodes solely on a single value (the value defined previously), a first part of the nodes are selected according to a first value (corresponding to a first criterion) and a second part of the nodes according to a second value (corresponds to another criterion or a plurality of other criteria). Among the various other criteria that can be considered, there is the path recommended by a pre-trained end-to-end learning algorithm, or a node that would have been selected by a cardiologist (then transformed into a numerical value by inverse reinforcement learning for example). Which results in the selection of the leaf node with the first highest value and of the leaf node with the second highest value, with the expansion of their 8 child nodes, with the recovery of the recompenses and the propagation of the values to the root of the tree.

FIG. 19 shows schematically an example of a first step of a third type of use of a tree search algorithm, in an automatic navigation method according to an embodiment of the invention.

The various nodes 10 are distributed in various levels, here only the root level 11 is shown. Its double default value is equal to 0-0 (first value of 0, and second value of 0).

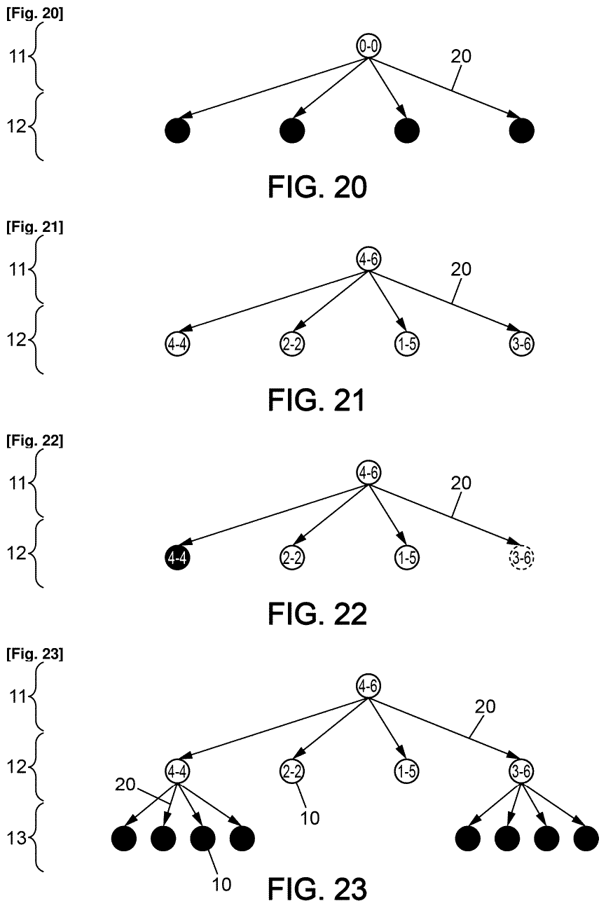

FIG. 20 shows schematically an example of a second step of a third type of use of a tree search algorithm, in an automatic navigation method according to an embodiment of the invention.

The various nodes 10 are distributed in various levels, the root level 11, the first filiation level 12 (also referred to as depth 1).

The passage between two nodes, of different filiations, and therefore from a filiation level of the parent type to a filiation level of the child type, for example from the root level 11 to the first filiation level 12, is an action 20 (to be selected from the 4 actions already presented which are: advancing or turning in one direction or turning in the other direction or retracting).

From the root node, the possible actions 20 are expanded, resulting in 4 new child nodes at the first filiation level 11.

FIG. 21 shows schematically an example of a third step of a third type of use of a tree search algorithm, in an automatic navigation method according to an embodiment of the invention.

Once the double values have been determined for these 4 new child nodes at the first filiation level 11, of this parent node that is the root node, namely from left to right the double values 4-4, 2-2, 1-5 and 3-6, the highest of these values, namely the first value 4 of the first node and the second value 6 of the fourth node (starting from the left in FIG. 21) of the first filiation level 12, are backpropagated:

> to its parent node, which is the root node, the double value of which then changes from 0-0 to 4-6.

FIG. 22 shows schematically an example of a fourth step of a third type of use of a tree search algorithm, in an automatic navigation method according to an embodiment of the invention.

The various nodes 10 are distributed in various levels, the root level 11, the first filiation level 12 (also referred to as depth 1).

The “leaf” nodes (i.e. the nodes that do not have any children in the tree) that are selected are:
firstly the node furthest to the left on FIG. 22, with the first highest value 4, with respect to a first criterion, since in fact the other leaf nodes all have first lower values, which are respectively 2, 1 and 3,
and secondly the node furthest to the right on FIG. 22, with the second highest value 6, with respect to a second criterion, since in fact that the other leaf nodes all have second lower values, which are respectively 4, 2 and 5.

FIG. 23 shows schematically an example of a fifth step of a third type of use of a tree search algorithm, in an automatic navigation method according to an embodiment of the invention.

From the two leaf nodes selected that are located at the first filiation level 12, firstly the node furthest to the left on FIG. 23, with a first highest value 4, and secondly the node furthest to the right on FIG. 23, with a second highest value 6, the possible actions 20 are expanded. This thus results in 8 new child nodes at the second filiation level 13, 4 new children per parent node previously selected at the first filiation level 12. The expansions from the two leaf nodes selected are implemented in parallel.

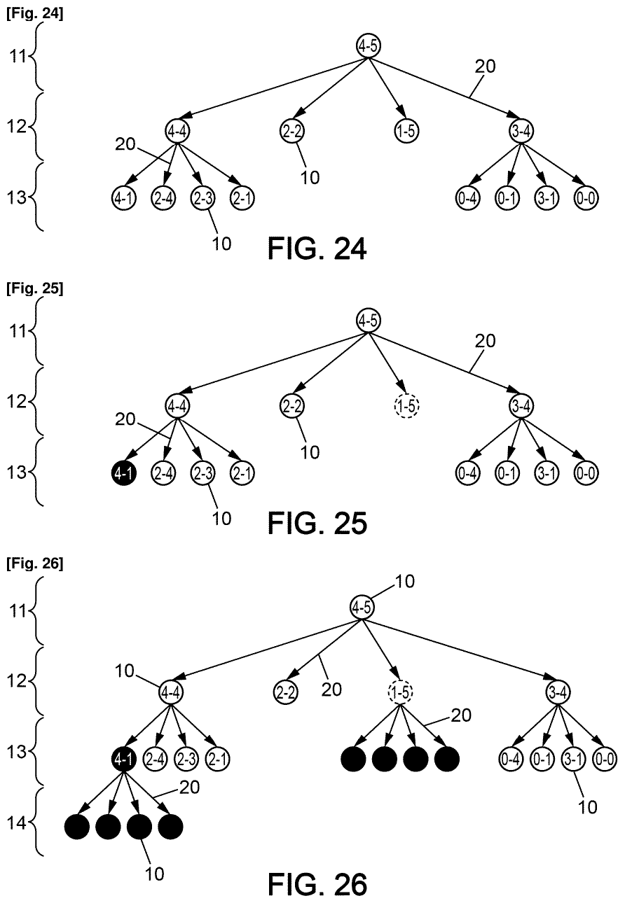

FIG. 24 shows schematically an example of a sixth step of a third type of use of a tree search algorithm, in an automatic navigation method according to an embodiment of the invention.

Once the double values have been determined for these 8 new child nodes at the second filiation level 13, namely, from left to right, the double values 4-1, 2-4, 2-3, 2-1, 0-4, 0-1, 3-1, and 0-0:
the highest of these first values, namely the first value 4 of the first node (starting from the left in FIG. 24) of the second filiation level 13, is backpropagated:
   to its parent node of the first filiation level 12, the first value of which remains at 4,
   to the node of the root level 11, the first value of which remains at 4,
the highest of these second values, namely the second value 4 of the fifth node (starting from the left in FIG. 24) of the second filiation level 13, is backpropagated:
   to its parent node of the first filiation level 12, the second value of which then changes from 6 to 4,
   but not to the node of the root level 11, the second value of which changes from 6 to 5, since the third node furthest to the left, of the first filiation level 12, has a second value at 5.

FIG. 25 shows schematically an example of a seventh step of a third type of use of a tree search algorithm, in an automatic navigation method according to an embodiment of the invention.

The leaf node that is selected for the first value is the leaf node located at the second filiation level 13, which is furthest to the left in FIG. 26, with a first highest value 4 (its double value being 4-1).

The leaf node that is selected for the second value is the leaf node located on the first filiation level 12, which is the third node furthest to the left on FIG. 25. This leaf node has in fact the second highest value 5 (its double value being 1-5), the other leaf nodes having second lower values that are respectively 1, 4, 3, 1, 2, 4, 1, 1, 0.

FIG. 26 shows schematically an example of an eighth step of a third type of use of a tree search algorithm, in an automatic navigation method according to an embodiment of the invention.

From the two leaf nodes selected that are located firstly at the second filiation level 13 for the first leaf node selected with the first highest value 4, and secondly at the first filiation level for the second leaf node selected with the second highest value 5, the possible actions 20 are expanded. This results in 8 new child nodes, including 4 new child nodes at the third filiation level 14 and 4 new child nodes at the second filiation level 13 (4 child nodes per parent node previously selected). The expansions from the two leaf nodes selected are implemented in parallel.

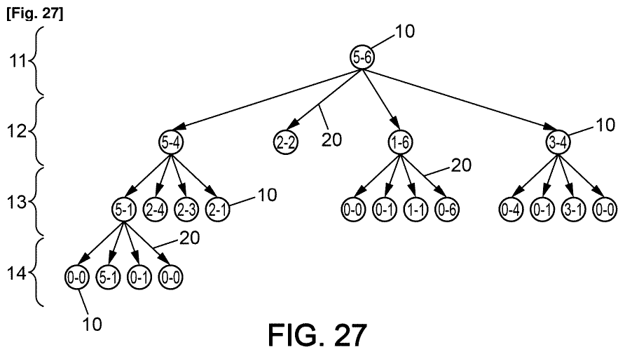

FIG. 27 shows schematically an example of a ninth step of a third type of use of a tree search algorithm, in an automatic navigation method according to an embodiment of the invention.

Once the double values have been determined for these 4 new child nodes at the third filiation level 14, namely from left to right, the double values 0-0, 5-1, 0-1 and 0-0:
the highest of the first values, namely the value 5 of the second node (starting from the left in FIG. 27) of the third filiation level 14, is backpropagated:
   to its parent node of the second filiation level 13, the double value of which then changes from 4-1 to 5-1,
   to its grandparent node of the first filiation level 12, the double value of which changes from 4-4 to 5-4,
   to the node of the root level 11, the first value of which changes from 4 to 5,
the highest of the second values, namely the value 1 of the second and third nodes (starting from the left in FIG. 27) of the third filiation level 14, is backpropagated solely to its parent node of the second filiation level 13 (the second value already being 1, it remains 1). This is because the second value of 1 is not backpropagated to the grandparent node (and therefore not to the root node either) since it is less than 4 (the node of double value 2-4 at the second filiation level 13 stops the backpropagation of the second value 1).

Likewise, once the double values have been determined for the 4 new child nodes at the second filiation level 13, namely from left to right the double values 0-0, 5-1, 0-1 and 0-0:
the highest of the first values, namely the value 1 of the seventh node (starting from the left in FIG. 27) of the second filiation level 13, is backpropagated solely to its parent node of the first filiation level 12, the double value of which becomes 1-6 (the first value already being 1, it remains 1). This first value of 1 is not backpropagated to the root node since the double value 5-4 of the first filiation level 12 blocks the backpropagation,
the highest of the second values, namely the value 6 of the fifth node (starting from the right in FIG. 27) of the second filiation level 13, is backpropagated:
   to its parent node of the first filiation level 12, the double value of which then changes from 1-5 to 1-6,
   to the node of the root level 11, the second value of which changes from 5 to 6.

Naturally the present invention is not limited to the examples and to the embodiment described and shown, but is capable of numerous variants accessible to a person skilled in the art. In particular, the number of actions 20 is not limited to 4. It is possible for example to increase the number of possible actions 20 by establishing the following actions: advancing, retracting, turning in a first direction at an angle of X°, turning in the first direction at an angle of Y°, turning in a second direction at an angle of X°, and turning in the second direction at an angle of Y°. The first direction of rotation being opposite to the second direction of rotation, and X being less than Y.

According to a possible embodiment, an additional action may be of implementing a command according to another algorithm for N time steps, for example an inverse command of a QP (standing for quadratic programming) constrained optimization algorithm. Another additional action may also be requesting the doctor to manipulate the instrument during N time steps.

1. Catheter robot comprising an automatic navigation system, for an elongate flexible medical instrument that is a catheter or a catheter guide, with free distal end, implementing an automatic navigation method comprising successively:
a step of creating a modeling:
of the elongate flexible medical instrument,
of a blood circulatory system of a patient, along which the elongate flexible medical instrument is intended to move,
of the interaction between this system and this elongate flexible medical instrument, by modeling the contact between this elongate flexible medical instrument and one or more walls of a blood vessel or vessels in this system,
from one or more images of the actual blood circulatory system—of this patient,
a step of determining:
the position of a start point in the modeled system,
the position of a point of arrival in the modeled system,
a path to be followed, by the elongate flexible medical instrument, between the start point and the point of arrival, by determining a path, along the modeled system, between the start point and the point of arrival, with preferentially obligatory passage points in the modeled system,
a step of planning a sequence of commands for movement of the elongate flexible medical instrument, obtained by a tree search procedure which, by using the modeling of the elongate flexible medical instrument and the modeling of said contact:
simulates various possible movements of the elongate flexible medical instrument in the modeled system,
evaluates the results of the simulation of these various possible movements,
selects the most promising simulated movements among these various possible movements, to best follow the determined path with respect to one or more given criteria,
a step of implementing the planned sequence of commands, along the actual blood circulatory system of said patient, with compensation for any differences with respect to the determined path along the modeled system, by closed-loop regulation.

2. The catheter robot comprising an automatic navigation system, for an elongate flexible medical instrument that is a catheter or a catheter guide, with free distal end, implementing the automatic navigation method comprising successively:
a step of creating a modeling:
of the elongate flexible medical instrument,
of a blood circulatory system of a patient, along which the elongate flexible medical instrument is intended to move,
of the interaction between this system and this elongate flexible medical instrument, by modeling the contact between this elongate flexible medical instrument and one or more walls of a blood vessel or vessels in this system,
from one or more images of the actual blood circulatory system of this patient,
a step of determining:
the position of a start point in the modeled system,
the position of a point of arrival in the modeled system,
a path to be followed, by the elongate flexible medical instrument, between the start point and the point of arrival, by determining a path, along the modeled system, between the start point and the point of arrival, with preferentially obligatory passage points in the modeled system,
a step of planning a sequence of commands for movement of the elongate flexible medical instrument, obtained by a tree search procedure which, by using the modeling of the elongate flexible medical instrument and the modeling of said contact:
simulates various possible movements of the elongate flexible medical instrument in the modeled system,
evaluates the results of the simulation of these various possible movements,
selects the most promising simulated movements among these various possible movements, to best follow the determined path with respect to one or more given criteria.

3. The catheter robot according to claim 1, wherein:
said automatic navigation method is able to be implemented in real time,
said tree search procedure of the planning step simultaneously implements in parallel, for a plurality of possible different movements of the elongate flexible medical instrument in the modeled system, for at least 2 or at least 3 or at least 4 possible different movements, the following planning cycle:
simulating a possible movement of the elongate flexible medical instrument in the modeled system,
evaluating the result of the simulation of this possible movement,
selecting the simulated movement, if the simulated movement is one of the most promising among the various possible movements, to best follow the determined path with respect to one or more given criteria.

4. The catheter robot according to claim 1, wherein,
if at the end of said implementation step one or more differences to be compensated for are evaluated as being too great, with respect to one or more given criteria:
then an adjustment of the modeling is made, and, on the adjusted modeling, the following are next implemented:
first a new step of determining a new path,
next a new step of planning a new sequence of commands for moving the elongate flexible medical instrument, from the new path determined,
finally, where applicable, a new step of implementing the new planned sequence of commands.

5. The catheter robot according to claim 1, wherein the step of creating modeling makes a representation of the physical system encompassing both the elongate flexible medical instrument and the blood circulatory system of a patient, along which the elongate flexible medical instrument is intended to move, by finite-element simulation.

6. The catheter robot according to claim 4, wherein:
the blood circulatory system of the patient is modeled:
either by point clouds,
or by meshings,
or by center lines associated with their respective diameters,
or by implicit surfaces.

7. The catheter robot according to claim 4, wherein:
the modeling of the blood circulatory system of the patient incorporates the movements affecting this blood circulatory system of the patient,
and preferentially incorporates the deformations of the heart of the patient when the heart is beating and/or the deformations caused by the breathing of the patient.

8. The catheter robot according to claim 1, wherein:
in said determination step:
said start point in the modeled system is positioned at the outlet of the guide catheter of the catheter robot, at the ostium,
and said point of arrival in the modeled system is positioned in the patient, at the lesion to be treated.

9. The catheter robot according to claim 1, wherein:
in said determination step:
the path to be followed is determined by interpolation between said start point and said point of arrival,
and preferentially by the use of a Dijkstra graph travel algorithm applied to the center lines of the blood vessels of the blood circulatory system.

10. The catheter robot according to claim 1, wherein said tree search procedure:
evaluates the results of the simulation of these various possible movements by attributing marks to each branch of the tree, and then calculating a value for each node in the tree from the marks attributed to the branches leading to this node,
selects the most promising simulated movements among these various possible movements, which are the simulated movements leading to the node for the highest value, to guide its exploration in the tree.

11. The catheter robot according to claim 10, wherein said tree search procedure:
attributes the marks in the following manner:
if the elongate flexible medical instrument advances along the determined path, then the corresponding possible movement of the elongate flexible instrument in the modeled system receives a positive mark,
if the elongate flexible medical instrument advances otherwise than along the determined path or if the elongate flexible medical instrument retracts or if the elongate flexible medical instrument stagnates along the determined path, then the corresponding possible movement of the elongate flexible medical instrument in the modeled system receives a zero or negative mark.

12. The catheter robot according to claim 10, wherein said tree search procedure:
attributes the marks in the following manner:
if the possible movement of the elongate flexible medical instrument along the determined path is considered to be similar to that which would result from the action of a doctor selected as a reference, then this possible movement receives a positive mark,
if the possible movement of the elongate flexible medical instrument along the determined path is considered to be different from that which would result from the action of a doctor selected as a reference, then this possible movement receives either a zero mark or a negative mark.

13. The catheter robot according to claim 10, wherein said tree search procedure:
partly attributes the marks in the following first manner:
if the elongate flexible medical instrument advances along the determined path, then the corresponding possible movement of the elongate flexible medical instrument in the modeled system receives a positive mark,
if the elongate flexible medical instrument advances otherwise than along the determined path or if the elongate flexible medical instrument retracts or if the elongate flexible medical instrument stagnates along the determined path, then the corresponding possible movement of the elongate flexible medical instrument in the modeled system receives a zero or negative mark,
also partly attributes the marks in the following second manner:
if the possible movement of the elongate flexible medical instrument along the determined path is considered to be similar to that which would result from the action of a doctor selected as a reference, then this possible movement receives a positive mark,
if the possible movement of the elongate flexible medical instrument—along the determined path is considered to be different from that which would result from the action of a doctor selected as a reference, then this possible movement receives either a zero mark or a negative mark,
uses, with preferentially predetermined weightings, optionally different from each other, respectively the first manner and the second manner.

14. The catheter robot according to claim 10, wherein the value of each node is calculated by totaling the marks of each branch leading to this node, or the value of each node is calculated in accordance with the UCB method.

15. The catheter robot according to claim 10, wherein said tree search procedure returns to pass through a node with a less good value, after a node with a better value has proved to be a dead end.

16. The catheter robot according to claim 1, wherein said step of implementing the planned sequence of commands is implemented by direct command implementing the movements of the actuators controlling the movements of the elongate flexible medical instrument.

17. The catheter robot according to claim 1, wherein said step of implementing the planned sequence of commands is implemented by inverse command calculating the movements of the actuators from the movements of the elongate flexible medical instrument.

18. The catheter robot according to claim 1, wherein, the catheter robot comprising an automatic navigation system for a plurality of elongate flexible medical instruments among which there are at least a catheter and a catheter guide associated with this catheter, these elongate flexible medical instruments being with a free distal end.

19. Automatic navigation system for an elongate flexible medical instrument that is a catheter or a catheter guide, of a catheter robot, with free distal end, implementing an automatic navigation method comprising successively:
a step of creating a modeling:
of the elongate flexible medical instrument,
of a blood circulatory system of a patient, along which the elongate flexible medical instrument is intended to move,
of the interaction between this system and this elongate flexible medical instrument, by modeling the contact between this elongate flexible medical instrument and one or more walls of a blood vessel or vessels in this system,
from one or more images of the actual blood circulatory system of this patient,
a step of determining:
the position of a start point in the modeled system,
the position of a point of arrival in the modeled system,
a path to be followed, by the elongate flexible medical instrument, between the start point and the point of arrival, by determining a path, along the modeled system, between the start point and the point of arrival, with preferentially obligatory passage points in the modeled system,
a step of planning a sequence of commands for movement of the elongate flexible medical instrument, obtained by a tree search procedure which, by using the modeling of the elongate flexible medical instrument and the modeling of said contact:
simulates various possible movements of the elongate flexible medical instrument in the modeled system,
evaluates the results of the simulation of these various possible movements,
selects the most promising simulated movements among these various possible movements, to best follow the determined path with respect to one or more given criteria,
a step of implementing the planned sequence of commands, along the actual blood circulatory system of said patient, with compensation for any differences with respect to the determined path along the modeled system, by closed-loop regulation.

20. Automatic navigation system for an elongate flexible medical instrument that is a catheter or a catheter guide, of a catheter robot, with free distal end, implementing an automatic navigation method comprising successively:
a step of creating a modeling:
of the elongate flexible medical instrument,
of a blood circulatory system of a patient, along which the elongate flexible medical instrument is intended to move,
of the interaction between this system and this elongate flexible medical instrument, by modeling the contact between this elongate flexible medical instrument and one or more walls of a blood vessel or vessels in this system,
from one or more images of the actual blood circulatory system of this patient,
a step of determining:
the position of a start point in the modeled system,
the position of a point of arrival in the modeled system,
a path to be followed, by the elongate flexible medical instrument, between the start point and the point of arrival, by determining a path, along the modeled system, between the start point and the point of arrival, with preferentially obligatory passage points in the modeled system,
a step of planning a sequence of commands for movement of the elongate flexible medical instrument, obtained by a tree search procedure which, by using the modeling of the elongate flexible medical instrument and the modeling of said contact:
simulates various possible movements of the elongate flexible medical instrument in the modeled system,
evaluates the results of the simulation of these various possible movements,
selects the most promising simulated movements among these various possible movements, to best follow the determined path with respect to one or more given criteria.

21-22. (canceled)
